(19) United States
(12) Patent Application Publication
(10) Pub. No.: US 2019/0151341 A1
(43) Pub. Date: May 23, 2019

(54) APPLICATION OF TENVERMECTIN FOR PREVENTING AND TREATING PARASITE INFECTION

(71) Applicant: ZHEJIANG HISUN PHARMACEUTICAL CO., LTD., Taizhou city, Zhejiang (CN)

(72) Inventors: Jidong WANG, Taizhou city, Zhejiang (CN); Jun HUANG, Taizhou city, Zhejiang (CN); Hui ZHANG, Taizhou city, Zhejiang (CN); Jiatan LIN, Taizhou city, Zhejiang (CN); Meihong LI, Taizhou city, Zhejiang (CN); Lingjian ZHANG, Taizhou city, Zhejiang (CN); Aiwen DENG, Taizhou city, Zhejiang (CN); Lingping WANG, Taizhou city, Zhejiang (CN)

(21) Appl. No.: 16/091,304

(22) PCT Filed: Mar. 31, 2017
(86) PCT No.: PCT/CN2017/079061

§ 371 Date: Oct. 4, 2018

(30) Foreign Application Priority Data
Apr. 7, 2016 (CN) 201610213645.2

(51) Int. Cl. A61K 31/7048; A61P 33/10; A61P 33/14

(52) U.S. Cl. CPC A61K 31/7048; A61P 33/14; A61P 33/10

(57) ABSTRACT

Disclosed herein are compounds tenvermectin A and B and mixtures of compounds tenvermectin A and B for preventing and treating human or animal parasitic infection. Tenvermectin A and B are macrolide compounds and are produced by genetically engineered bacteria. Tenvermectin A and B are structurally similar to avermectin, ivermectin, milbemycin and emamectin benzoate. The toxicities of tenvermectin A and B are significantly lower than avermectin, ivermectin, milbemycin and emamectin benzoate. The disclosed compounds and mixture of compounds belong to a class of compounds that are useful for killing and eliminating parasites, such as nematodes or arthropods.

### TECHNICAL FIELD

The present invention belongs to the field of medicine and particularly relates to the use of tenvermectin for preventing and treating parasites in human or animals.

### TECHNICAL BACKGROUND

Parasitic diseases, as diseases that are very harmful to livestock and poultry, often endanger animal health in a much hidden way. Most of parasitic diseases are chronic diseases and have no obvious symptoms in clinical practice, making livestock and poultry in a subclinical state for a long time, which is easy to be neglected by breeders. There are many types of animal parasitic diseases, which are widely distributed and mix infection is easily occurred. Once the diseases outbreak, they will seriously affect the animals' physical health and damage their reproductive ability, inhibit the growth and development of young animals, weaken the production performance of livestock and poultry, and reduce the quantity and quality of livestock and poultry products, causing serious economic losses to livestock production. Parasitic diseases not only cause harm to the animal husbandry industry, but also cause serious harm to human health. Parasitic zoonosis is one of the greatest enemies of human health, and it is a serious threat to public health.

As the development of animal husbandry and public health security requires, when fighting against parasitic diseases, the traditional medicines for the treatment of ectoparasites, such as trichlorfon, are highly toxic agents, thus are gradually eliminated, and the ideal anti-parasitic drugs, which are safe, broad-spectrum, high-efficient, residue-free or low-residue, with physicochemical properties of group-administration and low-cost will be on the stage.

Avermectins are a kind of natural and semi-synthetic antibiotics, which are produced by the fermentation of Streptomyces avermitilis. Avermectins have broad-spectrum anti-parasitic activities and can kill mature and immature nematodes and arthropods at a very low concentration. Although this kind of drugs has extremely low toxicities to bacteria, fungi, earthworm, plants and birds, they have higher toxic effects on certain aquatic organisms. After several weeks of administration of avermectins, there are drug residues in the animal feces, and the residual drugs in the agglomerated feces cannot be quickly decomposed. Therefore, avermectins are potentially harmful to aquatic organisms.

Patent No. CN201410208660.9 discloses a new class of macrolide compounds tenvermectin A and B (the structures thereof are shown in Formula I) produced by genetically engineered bacteria MA220 and its effect of preventing and treating Tetranychus cinnabarinus, Tetranychus urticae koch, Plutella xylostella, beet armyworm, Spodoptera litura, cotton bollworm, Agrotis ipsilon, wireworm, armyworm, pine caterpillar, pine wood nematode, rice stem borer and other pests and mites of agricultural and forestry crops.

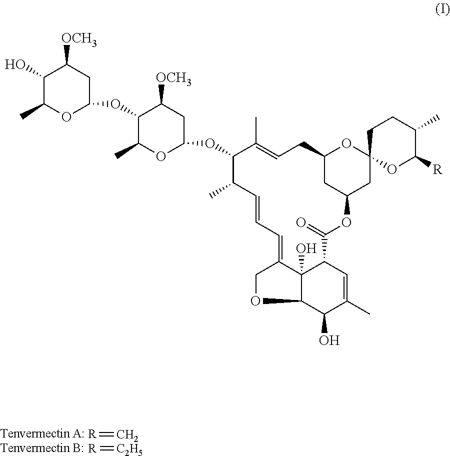

### SUMMARY OF THE INVENTION

The application provides the use of one compound or the mixture of two compounds of formula (I) below in the preparation of a medicament for preventing and treating human or animal parasites:

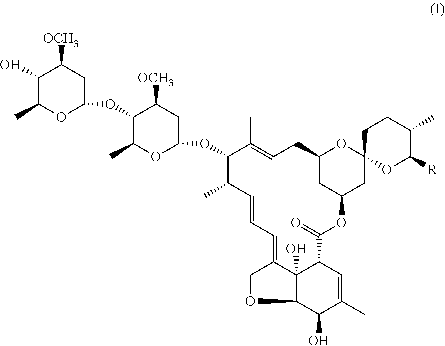

wherein R is selected from the group consisting of CH₃ or C₂H₅, the compound is tenvermectin A when R is —CH₃ and the compound is tenvermectin B when R is —C₂H₅.

In a preferred embodiment, the animal is selected from the group consisting of pigs, cattle, dogs, sheep, rabbits, chickens, ducks or gooses.

In a preferred embodiment, the parasite is selected from the group consisting of Nematoda, Insecta and Arachnida.

In a preferred embodiment, the parasite is selected from the group consisting of Strongylida, Rhabditia, Ascaridida, Oxyurata, Spirurida, Filarioidea, Aphasmidea, Diptera, Anoplura, Siphonaptera, Acarina or Acariforms, preferably, Strongylata, Ascaridida, Filarioidea, Aphasmidea, Acarina or Acariforms.

In a preferred embodiment, the parasite is selected from the group consisting of Trichostrongylidae, Ancylastomatidae, Oesophagostomum, Dictyocaulidae, Strongyloididae, Ascaridae, Toxocaridae, Ascaridiidae, Oxyuridae, Syphaciidae, Spiruridae, Filariata, Capillariidae, Trichinellidae, Trichuridae, Culicidae, Muscidae, Pediculidae, Pulicidae, Ixodidae or Sarcoptidae, preferably Ascaridae, Ascaridiidae, Trichuridae, Ixodidae or Sarcoptidae.

In a preferred embodiment, the parasite is selected from the group consisting of swine nematodes, Haematopinus, bovine ticks, nematodes in digestive track of sheep or Sarcoptes mites of sheep.

In a preferred embodiment, the compound of formula (I) is a mixture of tenvermectin A and tenvermectin B.

In a preferred embodiment, the animal is selected from the group consisting of fish, crustacean, mollusc or aquatic invertebrate, preferably grass carp, allogynogenetic crucian carp, silver carp, Pseudorasbora parva, mosquitofish, bighead carp, freshwater shrimp, river crab, Chinese mystery snail, Cyprinus carpio Songpu and ornamental fishes, etc., more preferably allogynogenetic crucian carp, grass carp, Cyprinus carpio Songpu or ornamental fishes.

In a preferred embodiment, the parasite is selected from the group consisting of sporozoan, cestode, nematode, Eimeriidae, Paragonimus, Schistosome, Dactylogyrus or Lernaea.

In a preferred embodiment, the compound of formula (I) is a mixture of tenvermectin A and tenvermectin B, preferably, the weight ratio of tenvermectin A to tenvermectin B in the mixture is ≥9:1, more preferably ≥19:1.

The inventors have surprisingly found that although tenvermectin A and tenvermectin B are structurally similar to avermectin, ivermectin, milbemycin and emamectin benzoate, the toxicities of tenvermectin A and tenvermectin B are significantly lower than those of similar products such as avermectin, ivermectin, milbemycin and emamectin benzoate through the toxicological experiments of zebrafish. Therefore, the product is greener and environmentally friendly and has better market application prospects.

The compound of formula (I) of the present invention can be prepared in the form of a conventional preparation. The conventional preparation forms include, for example, pour-on solution, tablet, injection and dry suspension.

### EMBODIMENTS

The application is further illustrated by the following examples, it must be stated that the examples are intended to illustrate the present invention and are not to be construed as limiting the present invention.

### Example 1

### Preparation of 1% Ivermectin, 1% Tenvermectin A and 1% Tenvermectin B Preparations

Ivermectin, tenvermectin A and tenvermectin B were all from Zhejiang Hisun Pharmaceutical Co., Ltd. As described in the ratios of the formulations in Table 1, the raw materials were dissolved in dimethylformamide, then were added in propylene glycol and were stirred evenly, and were aseptically filtered, 1% ivermectin, 1% tenvermectin A and 1% tenvermectin B injections were obtained respectively.

TABLE 1

| Preparations | Active ingredients | Excipient 1 | Excipient 2 |
|---|---|---|---|
| 1% Ivermectin | Ivermectin, 1 kg | Dimethylformamide, 10 L | Propylene glycol, |
| | | | 90 L |
| 1% Tenvermectin A | Tenvermectin A, | Dimethylformamide, 10 L | Propylene glycol, |
| | 1 kg | | 90 L |
| 1% | Tenvermectin B, | Dimethylformamide, 10 L | Propylene glycol, |
| Tenvermectin B | 1 kg | | 90 L |

### Example 2

Repelling Swine Nematode and Haematopinus by Tenvermectin A and Tenvermectin B

Test agents: 1% ivermectin injection, 1% tenvermectin A injection, and 1% tenvermectin B injection as obtained in Example 1.

Test animals and grouping: pigs cultured in the same farm were selected, and 40 pigs naturally infected with nematodes in the digestive tract were selected for testing through fecal examination. The average weight of the test pigs was 30-50 kg. Forty pigs were randomly divided into 4 groups, 10 for each group. The first group was the ivermectin control group, the second group was the tenvermectin A group, and the third group was the tenvermectin B group. All the medicated groups were injected subcutaneously in the neck according to 0.2 mg/kg·bw. The forth group was a blank group and was not administered.

Test Method:

1) The feces of the pigs in each group were collected and examined one by one a day before administration, and worm eggs were counted by the McMaster's method. The number of eggs per gram of feces (EPG) was counted and the condition of Haematopinus on the body surface was observed.

2) 1, 2, 3 and 4 weeks after administration, the feces of the pigs in each group were examined by the McMaster's method, and the EPG was counted and the changes of Haematopinus on the body surface were observed.

3) The negative conversion rates and egg reduction rates of the test pigs were calculated by a conventional method to assess the efficacy of repelling swine nematode.

4) The killing effects of swine Haematopinus of the agents were assessed according to the survival of swine Haematopinus on the 7^{th} day after administration.

Test Results:

1) It can be seen from Table 2 that the effect of repelling parasites of the tenvermectin A group, the tenvermectin B group and the ivermectin group were as follow: tenvermectin B group>tenvermectin A group>ivermectin group, and the efficacy of the tenvermectin B group and tenvermectin A group were more durable;

2) The experimental groups were administered until the 7^{th} day and in the following 3 weeks, the tenvermectin A group, the tenvermectin B group and the ivermectin group were observed, no active Haematopinus was found, and the blank group was observed, the amount of Haematopinus on the body surfaces of the pigs was not decreased.

TABLE 2
The negative conversion rate and egg reduction rate of the test pigs in each group

| | 1^{st} week | | 2^{nd} week | | 3^{rd} week | | 4^{th} week | |
|---|---|---|---|---|---|---|---|---|
| | Negative | Egg | Negative | Egg | Negative | Egg | Negative | Egg |
| | conversion | reduction | conversion | reduction | conversion | reduction | conversion | reduction |
| Group | rate (%) | rate (%) | rate (%) | rate (%) | rate (%) | rate (%) | rate (%) | rate (%) |
| Ivermectin | 70 | 76.2 | 90 | 91.2 | 80 | 100 | 60 | 66.2 |
| Tenvermectin A | 70 | 79.6 | 80 | 89.5 | 90 | 98.6 | 70 | 78.8 |
| Tenvermectin B | 80 | 85.3 | 100 | 100 | 100 | 100 | 90 | 95.8 |
| Blank | 0 | 0 | 0 | 0 | 0 | 0 | 0 | 0 |

### Example 3

Killing Effects of Tenvermectin A and Tenvermectin B on Bovine Ticks

Test agents: 1% ivermectin injection, 1% tenvermectin A injection and 1% tenvermectin B injection as obtained in Example 1.

Test animals and grouping: six cattle naturally infected with bovine ticks in the same village were selected for testing. The six cattle were randomly divided into three groups, two heads for each group. The first group was the ivermectin control group, the second group was the tenvermectin A group, and the third group was the tenvermectin B group. The medicated groups were injected subcutaneously according to 2 mg/kg·bw.

Test Methods:

1) The bovine ticks on the cattle in each group were counted 1 day before administration.

2) The bovine ticks on the cattle in each group were counted on the 1^{st}, 2^{nd} and 4^{th} week after administration.

3) The number of bovine ticks was calculated by a conventional method to assess the efficacy.

Test results: as shown in Table 3, the effects of repelling parasites of the tenvermectin A group, the tenvermectin B group and the ivermectin group were quite similar, but the efficacies of the tenvermectin A group and the tenvermectin B group were more durable.

TABLE 3
Number of ticks in each week after administration of the test cattle in each group

| | | | Number of ticks | Number of ticks after | | | |
|---|---|---|---|---|---|---|---|
| | | | one day before | administration (head) | | | |
| Group | Cattle | | administration | 1^{st} | 2^{nd} | 3nd | 4^{th} |
| No. | No. | Administration | (head) | week | week | week | week |
| 1 | 1 | Ivermectin | 35 | 0 | 0 | 1 | 0 |
| | 2 | Ivermectin | 22 | 0 | 0 | 0 | 1 |
| 2 | 3 | Tenvermectin A | 31 | 1 | 0 | 0 | 0 |
| | 4 | Tenvermectin A | 27 | 0 | 0 | 0 | 0 |
| 3 | 5 | Tenvermectin B | 25 | 0 | 0 | 0 | 0 |
| | 6 | Tenvermectin B | 32 | 0 | 0 | 0 | 0 |

### Example 4

Test of Tenvermectin A and Tenvermectin B in Repelling Nematode in Digestive Track of Sheep

Test agents: 1% ivermectin injection, 1% tenvermectin A injection, and 1% tenvermectin B injection as obtained in Example 1.

Test animals and grouping: sheep grazed under the same condition were selected, and 80 sheep of 0.8-2.0 years old naturally infected with digestive tract nematodes were selected through fecal examination. The average weight of the test sheep was 30 kg. The 80 sheep were randomly divided into 4 groups, 20 heads for each group. The first group was the ivermectin control group, the second group was the tenvermectin A group, and the third group was the tenvermectin B group. All the medicated groups were injected subcutaneously in the neck according to 0.2 mg/kg·bw. The fourth group was a blank group and was not medicated.

Test Methods:

1) The feces of the sheep in each group were collected and examined one by one a day before administration, and the worm eggs were counted by the McMaster's method, and the number of eggs per gram of feces (EPG) was counted.

2) On the 9^{th} day after administration, the sheep of each group were subjected to a fecal examination by McMaster's method, and EPG was counted.

3) The egg negative conversion rates and egg reduction rates were calculated according to a conventional method to assess the efficacy.

Test Results:

The detailed test results can be seen in Table 4. It can be seen from Table 4 that the effects of repelling parasites of the tenvermectin A group, the tenvermectin B group and the ivermectin group were quite similar, and the egg negative conversion rates and the egg reduction rates thereof were all 100%.

TABLE 4
The egg negative conversion rate and the egg reduction rate of sheep in each group

| | Number | | | | Egg |
|---|---|---|---|---|---|
| | of sheep | Negative | | | reduction |
| | negatively | conversion | EPG before | EPG after | rate |
| Group | conversed | rate (%) | administration | administration | (%) |
| Tenvermectin A | 20 | 100 | 11600 | 0 | 100 |
| Tenvermectin B | 20 | 100 | 12200 | 0 | 100 |
| Ivermectin | 20 | 100 | 9100 | 0 | 100 |
| Blank | 0 | 0 | 8600 | 9900 | 0 |

### Example 5

Treatment of Sarcoptes Mites of Sheep with Tenvermectin A and Tenvermectin B

Test agents: 1% ivermectin injection, 1% tenvermectin A injection and 1% tenvermectin B injection as obtained in Example 1.

Test animals and grouping: sheep grazed under the same conditions were selected. Upon parasitological examination, 40 sheep which had been infected with sheep Sarcoptes mites were selected. The 40 sheep were randomly divided into 4 groups, 10 heads for each group. The first group was the ivermectin control group, the second group was the tenvermectin A group, and the third group was the tenvermectin B group. All the medicated groups were injected subcutaneously in the neck according to 0.2 mg/kg·bw. The fourth group was a blank group and was not medicated.

Test methods: the changes of clinical symptoms were observed. The skin of sheep was scratched using a surgical blade with glycerin at the junction of the affected part and the healthy skin of the diseased sheep until the skin slightly bled. The skin scraps were taken then were placed in a dish and brought back to the laboratory. Then the skin scraps were placed on a slide, a drop of aqueous solution of 50% glycerol was added, and a cover slip was covered, and then the skin scraps were checked under a low magnification microscope. It was confirmed to be positive if live Sarcoptes mites were observed. Samples were taken before administration and at 1, 2, 3 and 4 weeks after administration to be examined. The samples were examined under a microscope, based on whether live mites were observed, the samples can be determined to be positive as long as one live mite was observed. Changes of clinical symptoms were observed as references.

Test results: the test results can be seen in Table 5. It can be seen from Table 5 that the tenvermectin A group and the tenvermectin B group have better treatment effects on Sarcoptes mites of sheep than the ivermectin group, and the advantageous of the effect of tenvermectin B was more obvious.

TABLE 5
Negative conversion number and negative conversion rate of sheep in each group

| | | 1^{st} week | | 2^{nd} week | | 3^{rd} week | | 4^{th} week | |
|---|---|---|---|---|---|---|---|---|---|
| | | Negative | Negative | Negative | Negative | Negative | Negative | Negative | Negative |
| | | conversion | conversion | conversion | conversion | conversion | conversion | conversion | conversion |
| Group | Administration | number | rate (%) | number | rate (%) | number | rate (%) | number | rate (%) |
| 1 | Ivermectin | 7 | 70 | 9 | 90 | 10 | 100 | 10 | 100 |
| 2 | Tenvermectin A | 8 | 80 | 9 | 90 | 10 | 100 | 10 | 100 |
| 3 | Tenvermectin B | 9 | 90 | 10 | 100 | 10 | 100 | 10 | 100 |
| 4 | Blank | 0 | 0 | 0 | 0 | 0 | 0 | 0 | 0 |

### Example 6

Test on the Toxicity of Tenvermectin to Zebrafish

Test agents: avermectin, milbemycin, ivermectin, tenvermectin A and tenvermectin B were all from Zhejiang Hisun Pharmaceutical Co., Ltd. The samples were formulated into 50 mg/ml mother liquors with DMF.

Test fish and water: zebrafish (Brachydanio rerio) was purchased from Zhejiang Academy of Agricultural Sciences, with the same size, average body length of 2-3 cm and average body weight of 0.3 g. The zebrafish was domesticated for 7 days indoors before the test. The natural mortality rate was zero. Feeding was stopped 1 day before the test and the fish were not fed during the test. The test water was tap water with residual chlorine removed by exposure to the sun for more than 24 hours, and the pH thereof was 6.8.

Test methods: semi-static method. Three level differences were provided for each sample: 0.5 ppm, 1.0 ppm and 1.5 ppm, and three parallel groups were provided for each level difference, 10 zebrafish were raised for each group, and blank controls (one group without agents and one group with only solvent) were provided. The corresponding volume of mother liquor was taken according to the concentration as required for each sample, and the volume of each sample was adjusted to 150 μl with DMF, and then each sample was added to the test group (containing 1.6 L of water). The room temperature was controlled at 22±2° C. for 96 hours, and the water was changed every 24 hours and the samples were re-added. The fish mortality rate was recorded for the first 8 hours and at 24, 48, 72 and 96 hours, and the dead fish were removed in time. Finally, the agents were divided into three grades according to the values of LC₅₀: low-toxic agents with a LC₅₀>10 ppm, middle-toxic agents with a LC₅₀ of 1.0-10 ppm, and high-toxic agents with a LC₅₀<1.0 ppm.

Test results: the test results can be seen in Table 6. It can be seen from Table 6 that the 96-hour survival rate of zebrafish was all greater than 50% when the concentration of tenvermectin A was 1 ppm, indicating that the 96-hour LC₅₀ of tenvermectin A on zebrafish was >1 ppm, tenvermectin A was middle-toxic; although tenvermectin B did not reach the level of middle toxic, its toxicity to zebrafish was much lower than those of avermectin, ivermectin, milbemycin and emamectin benzoate. When avermectin, ivermectin, milbemycin and emamectin benzoate were at 0.5 ppm, the 8-hour survival rates of zebrafish were all 0, indicating that the 96-hour LC₅₀ of avermectin, the 96-hour LC₅₀ of ivermectin, the 96-hour LC₅₀ of milbemycin and the 96-hour LC₅₀ of emamectin benzoate on zebrafish were <0.5 ppm, they were high-toxic.

TABLE 6
Tests on the toxicities of different agents to zebrafish

| | | Concen- | Survival condition | | | | |
|---|---|---|---|---|---|---|---|
| | | tration | (survival rates/%) | | | | |
| No. | Agent | (ppm) | 8 h | 24 h | 48 h | 72 h | 96 h |
| 1 | Tenvermectin A | 0.5 | 100 | 100 | 100 | 100 | 100 |
| | | 1.0 | 100 | 100 | 100 | 100 | 100 |
| | | 1.5 | 100 | 100 | 100 | 100 | 100 |
| 8 | Tenvermectin B | 0.5 | 100 | 70 | 50 | 0 | |
| | | 1.0 | 53 | 0 | | | |
| | | 1.5 | 10 | 0 | | | |
| 9 | Avermectin | 0.5 | 0 | | | | |
| | | 1.0 | 0 | | | | |
| | | 1.5 | 0 | | | | |
| 10 | Milbemycin | 0.5 | 0 | | | | |
| | | 1.0 | 0 | | | | |
| | | 1.5 | 0 | | | | |
| 11 | Ivermectin | 0.5 | 0 | | | | |
| | | 1.0 | 0 | | | | |
| | | 1.5 | 0 | | | | |
| 12 | Methylamino | 0.5 | 0 | | | | |
| | avermectin | 1.0 | 0 | | | | |
| | benzoate | 1.5 | 0 | | | | |
| | (Emamectin | | | | | | |
| | benzoate) | | | | | | |

### Example 7

Test on the Toxicity of Tenvermectin to Allogynogenetic Crucian Carp

Allogynogenetic crucian carp is one of the main aquaculture species in Jiangsu and Zhejiang provinces. In many areas, the production of allogynogenetic crucian carp is at least 30% of freshwater fish production. Therefore, it is of great significance for the rational use of drugs in aquaculture production to understand the toxic effect of tenvermectin A on allogynogenetic crucian carp.

Test fish and water: allogynogenetic crucian carp was purchased from Taowang Farm in Jiaojiang District, Taizhou City, with a body length of (9.60±0.52) cm and a body weight of (28.22±3.44) g. The healthy and vigorous fish with the same size were selected and temporarily raised in an indoor aquarium. They were domesticated for 7 days indoors before the test. The natural mortality rate was zero. Feeding was stopped 1 day before the test and the fish were not fed during the test. The test water was tap water with residual chlorine removed by exposure to the sun for more than 24 hours, and the pH thereof was 6.8.

Test Agents:

99.4% Tenvermectin A (the mass content of tenvermectin B was 0.02%) (Zhejiang Hisun Pharmaceutical Co., Ltd.);

Tenvermectin (the mass ratio of tenvermectin A/tenvermectin B was 95/5) (Zhejiang Hisun Pharmaceutical Co., Ltd.);

Tenvermectin (the mass ratio of tenvermectin A/tenvermectin B was 90/10) (Zhejiang Hisun Pharmaceutical Co., Ltd.);

Tenvermectin (the mass ratio of tenvermectin A/tenvermectin B was 85/15) (Zhejiang Hisun Pharmaceutical Co., Ltd.);

99.1% Tenvermectin B (the mass content of tenvermectin A was 0.51%) (Zhejiang Hisun Pharmaceutical Co., Ltd.);

92% Avermectin (Zhejiang Qianjiang Biochemical Co., Ltd.);

96% Ivermectin (Zhejiang Hisun Pharmaceutical Co., Ltd.);

90% Methylamino avermectin benzoate (Emamectin benzoate) (Zhejiang Shenghua Biok Biotechnology Co., Ltd.).

The samples were formulated into 50 mg/ml mother liquors with DMF.

Method: semi-static method. Three level differences were provided for each sample: 0.5 ppm, 1.0 ppm and 2.0 ppm, and three parallel groups were provided for each level difference, 10 zebrafish were raised for each group, and blank controls (one group without agents and one group with only solvent) were provided. The corresponding volume of mother liquor was taken according to the concentration as required for each sample, and the volume of each sample was adjusted to 150 μl with DMF, and then each sample was added to the test group (containing 1.6 L of water). The room temperature was controlled at 22±2° C. for 96 hours, and the water was changed every 24 hours and the samples were re-added. The fish mortality rate was recorded for the first 8 hours and at 24, 48, 72 and 96 hours, and the dead fish were removed in time. Finally, the agents were divided into three grades according to the values of LC₅₀: low-toxic agents with a LC₅₀>10 ppm, middle-toxic agents with a LC₅₀ of 1.0-10 ppm, and high-toxic agents with a LC₅₀<1.0 ppm. The test results can be seen in Table 7.

TABLE 7
Tests on the toxicities of different agents
to allogynogenetic crucian carp

| | | Concen- | Survival condition | | | | |
|---|---|---|---|---|---|---|---|
| | | tration | (survival rates/%) | | | | |
| No. | Agent | (ppm) | 8 h | 24 h | 48 h | 72 h | 96 h |
| 1 | 99.4% | 0.5 | 100 | 100 | 100 | 100 | 100 |
| | Tenvermectin A | 1.0 | 100 | 100 | 100 | 100 | 100 |
| | | 2.0 | 100 | 100 | 100 | 100 | 100 |
| 2 | Tenvermectin | 0.5 | 100 | 100 | 100 | 100 | 100 |
| | A/B = 95:5 | 1.0 | 100 | 100 | 100 | 100 | 100 |
| | | 2.0 | 100 | 100 | 100 | 100 | 100 |
| 3 | Tenvermectin | 0.5 | 100 | 100 | 100 | 100 | 100 |
| | A/B = 90:10 | 1.0 | 100 | 100 | 93 | 83 | 60 |
| | | 2.0 | 87 | 60 | 33 | 13 | 0 |
| 4 | Tenvermectin | 0.5 | 100 | 100 | 100 | 100 | 100 |
| | A/B = 85:15 | 1.0 | 100 | 83 | 63 | 47 | 17 |
| | | 2.0 | 77 | 43 | 10 | 0 | |
| 8 | 99.1% | 0.5 | 100 | 80 | 33 | 0 | |
| | Tenvermectin B | 1.0 | 75 | 3 | | | |
| | | 2.0 | 20 | 0 | | | |
| 9 | 92% | 0.5 | 0 | | | | |
| | Avermectin | 1.0 | 0 | | | | |
| | | 2.0 | 0 | | | | |
| 10 | 96% | 0.5 | 0 | | | | |
| | Ivermectin | 1.0 | 0 | | | | |
| | | 2.0 | 0 | | | | |
| 11 | 90% | 0.5 | 0 | | | | |
| | Methylamino | 1.0 | 0 | | | | |
| | avermectin | 2.0 | 0 | | | | |
| | benzoate | | | | | | |
| | (Emamectin | | | | | | |
| | benzoate) | | | | | | |

The results showed that 99.4% tenvermectin A and tenvermectin A:B=95:5 were low toxic to allogynogenetic crucian carp, the 96-hour survival rate of zebrafish was still greater than 50% when the concentration of tenvermectin A:B=90:10 was 1 ppm, indicating that the 96-hour LC₅₀ of tenvermectin A:B=90:10 on allogynogenetic crucian carp was >1 ppm, tenvermectin A:B=90:10 was middle-toxic; and the toxicity of tenvermectin to allogynogenetic crucian carp decreased as the proportion of tenvermectin A increased. When avermectin, ivermectin, milbemycin and emamectin benzoate were at 0.5 ppm, the 8-hour survival rates of allogynogenetic crucian carp were all 0, indicating that the 96-hour LC₅₀ of avermectin, the 96-hour LC₅₀ of ivermectin and the 96-hour LC₅₀ of emamectin benzoate on allogynogenetic crucian carp were <0.5 ppm, they were high-toxic.

### Example 8

### Efficacy Test of Tenvermectin for Preventing and Treating Lernaea

The efficacy test was carried out in a laboratory next to the fish pond where Lernaea parasitic disease outbroke near the Changtan Reservoir in Huangyan District, Taizhou City. Test water was from the fish pond. The experimental container was a plastic aquarium of 85 cm×45 cm×35 cm. Each aquarium stored 100 L of pool water, water temperature was (27.0±0.5) ° C., pH was 7.4-7.8, and it was inflated continuously.

Test Agents:

92% Avermectin (Zhejiang Qianjiang Biochemical Co., Ltd.);

96% Ivermectin (Zhejiang Hisun Pharmaceutical Co., Ltd.);

95% Tenvermectin A (Zhejiang Hisun Pharmaceutical Co., Ltd.);

92% Tenvirmectin B (Zhejiang Hisun Pharmaceutical Co., Ltd.).

In the laboratory, the above-mentioned original medicines were respectively formulated into the following preparations for later use: 1.8% avermectin emulsifiable concentrate, 1.8% ivermectin emulsifiable concentrate, and 1.8% tenvermectin emulsifiable concentrate A (single component: tenvermectin A, wherein the content of impurity: tenvermectin B was 0.02%), 1.8% tenvermectin emulsifiable concentrate B (tenvermectin A:tenvermectin B=9:1 (weight ratio, the same hereinafter)), 1.8% tenvermectin emulsifiable concentrate C (single component: tenvermectin B, wherein the content of impurity: tenvermectin A was 0.51%).

The dosages of 1.8% tenvermectin emulsifiable concentrates A, B, C and 1.8% avermectin emulsifiable concentrate and 1.8% ivermectin emulsifiable concentrate were 0.01 mg/L, 0.02 mg/L and 0.04 mg/L, respectively. The liquids were not changed during the test. Two diseased allogynogenetic crucian carp were placed in each aquarium, and the number of Lernaea attached to each test fish was counted before the fish were placed. Each test was performed with clear water as a control, and each solution concentration was repeated 6 times. After the experiment started, observation was continuously conducted for the first 6 hours, and then their behavior, whether there were poisoning symptoms and a decrease of the population of parasites were observed aperiodicity. The number of residual Lernaea of each test group was recorded at 6 h, 12 h, 24 h, 48 h, and 96 h. The survival rate of parasitic Lernaea population on the body surface of allogynogenetic crucian carp after administration can be seen in Table 8. The corrective efficacy was calculated using the formula of Henderson-Tilton, and the significance of the difference was analyzed by the single factor method LSD test according to the standard of a=0.05 (0.01).

$$Corrective efficacy ( \% ) = \frac{\begin{matrix}Survival rate in control area - Survival rate \\ in prevention and treatment area\end{matrix}}{Survival rate in control area} \times 100 \%$$

TABLE 8
The survival rate of parasitic Lernaea population on the body
surface of allogynogenetic crucian carp after administration

| | | Concen- | Survival condition | | | | |
|---|---|---|---|---|---|---|---|
| | | tration | (survival rate/%) | | | | |
| No. | Agent | (ppm) | 6 h | 12 h | 24 h | 48 h | 96 h |
| 1 | 1.8% | 0.01 | 48 | 26 | 17 | 8 | 3 |
| | Tenvermectin | 0.02 | 32 | 18 | 12 | 4 | 0 |
| | emulsifiable | 0.04 | 22 | 14 | 5 | 0 | 0 |
| | concentrate A | | | | | | |
| 2 | 1.8% | 0.01 | 41 | 22 | 11 | 6 | 2 |
| | Tenvermectin | 0.02 | 30 | 16 | 9 | 2 | 0 |
| | emulsifiable | 0.04 | 21 | 12 | 2 | 0 | 0 |
| | concentrate B | | | | | | |
| 3 | 1.8% | 0.01 | 36 | 18 | 9 | 4 | 0 |
| | Tenvermectin | 0.02 | 22 | 11 | 2 | 0 | 0 |
| | emulsifiable | 0.04 | 16 | 6 | 0 | 0 | 0 |
| | concentrate C | | | | | | |
| 4 | 1.8% | 0.01 | 63 | 42 | 30 | 22 | 10 |
| | Avermectin | 0.02 | 58 | 40 | 26 | 16 | 8 |
| | emulsifiable | 0.04 | 46 | 28 | 20 | 15 | 5 |
| | concentrate | | | | | | |
| 5 | 1.8% | 0.01 | 60 | 38 | 26 | 18 | 6 |
| | Ivermectin | 0.02 | 53 | 31 | 23 | 12 | 4 |
| | emulsifiable | 0.04 | 40 | 22 | 16 | 6 | 0 |
| | concentrate | | | | | | |
| 6 | Control | | 100 | 100 | 100 | 100 | 100 |

The relative controlling efficacies after administration of 0.02 mg/L agents for 24 hours can be seen in Table 9.

TABLE 9
The relative controlling efficacies after administration
of 0.02 mg/L agents for 24 hours

| | Survival | | | |
|---|---|---|---|---|
| | rate of | | Significance | |
| | parasite | Relative | of | |
| | population | controlling | difference* | |
| Test agent | (%) | efficacy (%) | 5% | 1% |
| 1.8% Avermectin emulsifiable | 24.50 | 75.50 | b | B |
| concentrate | | | | |
| 1.8% Ivermectin emulsifiable | 23.50 | 76.50 | b | B |
| concentrate | | | | |
| 1.8% Tenvermectin emulsifiable | 4.50 | 95.50 | a | A |
| concentrate A | | | | |
| 1.8% Tenvermectin emulsifiable | 3.83 | 96.17 | a | A |
| concentrate B | | | | |
| 1.8% Tenvermectin emulsifiable | 4.00 | 96.00 | a | A |
| concentrate C | | | | |
| Control | 100 | — | — | — |

*Wherein the same letter in the column of significance of difference indicates no significant difference.

It can be seen from Table 9 that when tenvermectin A and tenvermectin B were mixed in different weight ratios (single component: tenvermectin A, tenvermectin A:tenvermectin B=9:1, single component: tenvermectin B), there were significant controlling effects on the parasitic Lernaea on the body surface of allogynogenetic crucian carp, and the controlling efficacies were all above 90%, which were superior to avermectin and ivermectin of the same dosage.

Use of tenvermectin of the present invention has been described by specific examples, and those skilled in the art can learn from the contents of the present invention, appropriately change the raw materials, process conditions and the like to achieve corresponding other purposes, and the related changes are not deviated from the content of the present invention. All similar substitutions and modifications are obvious to those skilled in the art and are considered to be included within the scope of the present invention.

1. A method of preventing and treating human or animal parasites, the method comprising:
administering one compound or a mixture of two compounds of formula (I) to a human or animal: 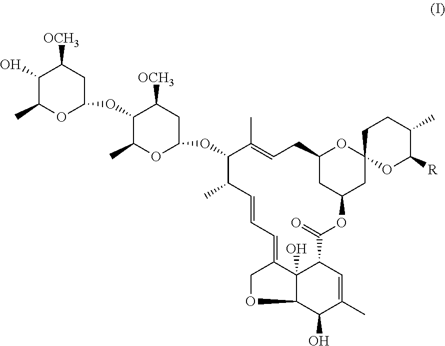
wherein R is selected from the group consisting of CH₃ and C₂H₅,
wherein the compound is tenvermectin A when R is —CH₃ and the compound is tenvermectin B when R is —C₂H₅.

2. The method according to claim 1, wherein the animal is selected from the group consisting of pigs, cattle, dogs, sheep, rabbits, chickens, ducks, geese, and any combination thereof.

3. The method according to claim 1, wherein the parasite is selected from the group consisting of Nematoda, Insecta, Arachnida, and any combination thereof.

4. The method according to claim 1, wherein the parasite is selected from the group consisting of Strongylida, Rhabditia, Ascaridida, Oxyurata, Spirurida, Filarioidea, Aphasmidea, Diptera, Anoplura, Siphonaptera, Acarina, Acariforms, and any combination thereof.

5. The method according to claim 1, wherein the parasite is selected from the group consisting of Trichostrongylidae, Ancylastomatidae, Oesophagostomum, Dictyocaulidae, Strongyloididae, Ascaridae, Toxocaridae, Ascaridiidae, Oxyuridae, Syphaciidae, Spiruridae, Filariata, Capillariidae, Trichinellidae, Trichuridae, Culicidae, Muscidae, Pediculidae, Pulicidae, Ixodidae Sarcoptidae, and any combination thereof.

6. The method according to claim 1, wherein the parasite is selected from the group consisting of swine nematode, Haematopinus, bovine ticks, nematodes in digestive track of sheep Sarcoptes mites of sheep, and any combination thereof.

7. The method according to claim 1, wherein tenvermectin A or tenvermectin B are administered to a human or an animal.

8. The method according to claim 1, wherein the animal is selected from the group consisting of fish, crustacean, mollusk, aquatic invertebrate, and any combination thereof.

9. The method according to claim 8, wherein the parasite is selected from the group consisting of sporozoan, cestode, nematode, Eimeriidae, Paragonimus, Schistosome, Dactylogyrus, Lernaeidae, and any combination thereof.

10. The method according to claim 8, wherein a mixture of tenvermectin A or tenvermectin B is administered to a human or an animal.

11. The method according to claim 4, wherein the parasite is selected from the group consisting of Strongylata, Ascaridida, Filarioidea, Aphasmidea, Acarina, Acariforms, and any combination thereof.

12. The method according to claim 5, wherein the parasite is selected from the group consisting of Ascaridae, Ascaridiidae, Trichuridae, Ixodidae Sarcoptidae, and any combination thereof.

13. The method according to claim 8, wherein the animal is selected from the group consisting of grass carp, allogynogenetic crucian carp, silver carp, Pseudorasbora parva, mosquitofish, bighead carp, freshwater shrimp, river crab, Chinese mystery snail, Cyprinus carpio Songpu, ornamental fishes, and any combination thereof.

14. The method according to claim 13, wherein the animal is selected from the group consisting of allogynogenetic crucian carp, grass carp, Cyprinus carpio Songpu ornamental fishes, and any combination thereof.

15. The method according to claim 10, wherein a weight ratio of tenvermectin A to tenvermectin B is ≥9:1.

16. The method according to claim 15, wherein a weight ratio of tenvermectin A to tenvermectin B is ≥19:1.
